(19) United States
(12) Patent Application Publication
(10) Pub. No.: US 2015/0297565 A1
(43) Pub. Date: Oct. 22, 2015

(54) PHENYLPYRAZOLE INJECTABLE COMPOSITIONS

(71) Applicant: Zoetis Services LLC, Florham Park, NJ (US)

(72) Inventors: Guy F. DeRose, Kalamazoo, MI (US); Stephen L. Secreast, Kalamazoo, MI (US); Telma Moreira da Silva, Guarulhos (BR)

(73) Assignee: Zoetis Services LLC, Florham, NJ (US)

(21) Appl. No.: 14/438,773

(22) PCT Filed: Dec. 11, 2013
(86) PCT No.: PCT/US13/74374

§ 371 Date: Apr. 27, 2015

Related U.S. Application Data
(60) Provisional application No. 61/736,203, filed on Dec. 12, 2012.

(51) Int. Cl. A61K 31/415

(52) U.S. Cl. CPC A61K 31/415

(57) ABSTRACT

This invention relates to a veterinary composition comprising a fluorinated cyclopropylphenylpyrazole. The veterinary composition also comprises a veterinary acceptable carrier including a triglyceride, a solvating agent and optionally an antioxidant. The compositions are injectable. The invention also relates to a method of treating an animal with a parasitic infestation by administering the veterinary composition to the animal in need thereof.

### FIELD OF INVENTION

This invention relates to a novel antiparasiticidal composition comprising a fluorinated cyclopropylphenylpyrazole, at least one veterinary acceptable carrier, and optionally, an antioxidant, and a method of treating an animal with a parasitic infestation with said veterinary composition.

### BACKGROUND OF THE INVENTION

The present invention relates to a new veterinary composition comprising a fluorinated cyclopropylphenylpyrazole for treating an animal with a parasitic infection or infestation, particularly an ectoparasitic infection or infestation. The fluorinated cyclopropylphenylpyrazoles of the instant invention were originally disclosed within a broad genus of compounds in WO1998/24767 and were subsequently exemplified in WO2005/060749. The present invention provides an improved injectable veterinary composition to that described in the prior art.

The compounds currently available for parasitic treatment of animals do not always demonstrate good activity, good speed of action, or a long duration of action. Most treatments contain hazardous chemicals that can have serious consequences, including lethality from accidental ingestion. Persons applying these agents are generally advised to limit their exposure. Pet collars and tags have been utilized to overcome some problems, but these are susceptible to chewing, ingestion, and subsequent toxicological affects to the animal. Thus, current treatments achieve varying degrees of success which depend partly on toxicity, method of administration, and efficacy. Currently, some agents are actually becoming ineffective due to parasitic resistance. Hence, there is a need for a stable and effective antiparasitic composition.

The veterinary composition of the present invention provides an improved pharmacokinetic profile which leads to improved clinical ectoparasitic efficacy than other known phenylpyrazoles, for example, fipronil.

### SUMMARY OF THE INVENTION

The antiparasitic compositions of the present invention may be used to prevent, treat, and control acarids (for example, ticks, and mites) and insect (for example, fleas, mosquitos, sandflies, and other flies) infestation in animals. In addition, the invention contemplates the control and prevention of tick borne diseases (for example, bovine anaplasmosis and babesiosis, epizootic bovine abortion, and theileriosis), insect born diseases (for example, leishmaniasis), and mite born diseases (for example, demidicosis). Thus, according to the present invention, there is provided a novel injectable composition.

The present invention relates to a novel veterinary composition comprising a fluorinated cyclopropylphenylpyrazole compound, stereoisomers thereof, or a veterinary acceptable salt thereof, at least one acceptable carrier, and optionally, at least one antioxidant.

In another aspect of the present invention, the veterinary composition comprises a fluorinated cyclopropylphenylpyrazole compound, stereoisomers thereof, or a veterinary acceptable salt thereof, at least one acceptable carrier which consists of a triglyceride, solvating agent, or mixture thereof, and optionally, at least one antioxidant.

In another aspect of the present invention, the veterinary composition comprises a fluorinated cyclopropylphenylpyrazole compound, stereoisomers thereof, or a veterinary acceptable salt thereof, at least one triglyceride, at least one solvating agent, or mixture thereof, and at least one antioxidant.

In another aspect of the present invention, the veterinary composition comprises a fluorinated cyclopropylphenylpyrazole compound, stereoisomers thereof, or a veterinary acceptable salt thereof, a triglyceride, a solvating agent, and at least one antioxidant.

In another aspect of the present invention, the veterinary composition comprises a fluorinated cyclopropylphenylpyrazole compound, stereoisomers thereof, or a veterinary acceptable salt thereof, a triglyceride, a solvating agent, and an antioxidant.

In another aspect of the invention, the fluorinated cyclopropylphenylpyrazole compound is selected from the group consisting of 5-amino-1-[2,6-dichloro-4-(trifluoromethyl)phenyl]-4-[2,2-difluoro-1-(trifluoromethyl)cyclopropyl]-1H-pyrazole-3-carbonitrile; 5-amino-1-[2,6-dichloro-4-(trifluoromethyl)phenyl]-4-[1-(difluoromethyl)-2,2-difluorocyclopropyl]-1H-pyrazole-3-carbonitrile; 5-amino-4-{1-[chloro(fluoro)methyl]-2,2-difluorocyclopropyl}-1-[2,6-dichloro-4-(trifluoro-methyl)phenyl]-1H-pyrazole-3-carbonitrile; 5-amino-1-[2,6-dichloro-4-(trifluoro-methyl)phenyl]-4-[1-(difluoromethyl)-2,2,3,3-tetrafluorocyclopropyl]-1H-pyrazole-3-carbonitrile; and 5-amino-1-[2,6-dichloro-4-(trifluoromethyl)phenyl]-4-[2,2,3,3-tetrafluoro-1-(trifluoromethyl)cyclopropyl]-1H-pyrazole-3-carbonitrile; stereoisomers thereof, or a veterinary acceptable salt thereof. In yet another aspect of the invention, the fluorinated cyclopropylphenylpyrazole compound is 5-amino-1-[2,6-dichloro-4-(trifluoromethyl)phenyl]-4-[2,2-difluoro-1-(trifluoromethyl)-cyclopropyl]-1H-pyrazole-3-carbonitrile (Compound 1); stereoisomers thereof, or a veterinary acceptable salt thereof.

In yet another aspect of the invention, the triglyceride is selected from the group consisting of cottonseed oil, castor oil, sesame oil, linseed oil, safflower oil, peanut oil, and soybean oil, or a mixture thereof. In yet another aspect of the invention, the triglyceride is selected from the group consisting of cottonseed oil, castor oil, and sesame oil, or a mixture thereof. In yet another aspect of the invention, the triglyceride is cottonseed oil.

In yet another aspect of the invention, the solvating agent is selected from the group consisting of ethyl oleate, caprylic/capric triglyceride, caprylic/capric/linoleic triglyceride, caprylic/capric/succinic triglyceride, propylene glycol dicaprylate/dicaprate, triacetin, benzyl benzoate, or a mixture thereof. In yet another aspect of the invention, the solvating agent is selected from the group consisting of ethyl oleate, triacetin, and propylene glycol dicaprylate/dicaprate, or a mixture thereof. In yet another aspect of the invention, the solvating agent is propylene glycol dicaprylate/dicaprate.

In yet another aspect of the invention, the antioxidant is selected from the group consisting of butylated hydroxyanisole or butylated hydroxytoluene, or a mixture thereof. In yet another aspect of the invention, the antioxidant is butylated hydroxyanisole. In yet another aspect of the invention, the antioxidant is butylated hydroxytoluene.

In yet another aspect of the invention, the veterinary composition further comprises at least one co-solvent. In yet another aspect of the invention, the co-solvent is selected from the group consisting of ethanol, a di-glycol monoalkyl ether, benzyl alcohol, and mixtures thereof. In yet another aspect of the invention, the co-solvent is selected from the group consisting of ethanol, a di-glycol monoalkyl ether, and benzyl alcohol. In yet another aspect of the invention, the co-solvent is ethanol. In yet another aspect of the invention, the co-solvent is a di-glycol monoalkyl ether. In yet another aspect of the invention, the co-solvent is benzyl alcohol.

In yet another aspect of the invention, the veterinary composition comprises a) 5-amino-1-[2,6-dichloro-4-(trifluoromethyl)phenyl]-4-[2,2-difluoro-1-(trifluoromethyl)-cyclopropyl]-1H-pyrazole-3-carbonitrile; stereoisomer thereof, or a veterinary acceptable salt thereof, b) a triglyceride, c) a solvating agent, and optionally, d) an antioxidant. In yet another aspect of the invention, the veterinary composition comprises a) 5-amino-1-[2,6-dichloro-4-(trifluoromethyl)phenyl]-4-[2,2-difluoro-1-(trifluoromethyl)-cyclopropyl]-1H-pyrazole-3-carbonitrile; stereoisomer thereof, or a veterinary acceptable salt thereof, b) a triglyceride, c) a solvating agent, and d) an antioxidant. In yet another aspect of the invention, the veterinary composition comprises a) 5-amino-1-[2,6-dichloro-4-(trifluoromethyl)phenyl]-4-[2,2-difluoro-1-(trifluoromethyl)-cyclopropyl]-1H-pyrazole-3-carbonitrile; stereoisomer thereof, or a veterinary acceptable salt thereof, b) a triglyceride, c) a solvating agent, d) an antioxidant, and optionally, e) a co-solvent. In yet another aspect of the invention, the veterinary composition comprises a) 5-amino-1-[2,6-dichloro-4-(trifluoromethyl)phenyl]-4-[2,2-difluoro-1-(trifluoromethyl)-cyclopropyl]-1H-pyrazole-3-carbonitrile; stereoisomer thereof, or a veterinary acceptable salt thereof, b) a triglyceride, c) a solvating agent, d) an antioxidant, and e) a co-solvent.

In another aspect of the present invention is a method of treating an animal with a parasitic infection or infestation by administering an effective amount of the veterinary composition to the animal in need thereof. In yet another aspect of the present invention is a method of treating an animal with an ectoparasitic infection or infestation by administering an effective amount of the veterinary composition to the animal in need thereof.

In another aspect of the present invention, the veterinary acceptable composition is administered by injection. In another aspect of the present invention, the veterinary acceptable composition is administered by subcutaneous injection.

In another aspect of the present invention, the amount of the fluorinated cyclopropyl phenylpyrazole is administered to the animal at a dose of about 2 mg/kg.

### DEFINITIONS

For purposes of the present invention, as described and claimed herein, the following terms and phrases are defined as follows:

“About” when used in connection with a measurable numerical variable, refers to the indicated value of the variable and to all values of the variable that are within the experimental error of the indicated value (e.g., within the 95% confidence interval for the mean) or within 10 percent of the indicated value, whichever is greater.

“Animal” as used herein, unless otherwise indicated, refers to an individual animal, and said individual animal is a mammal or fish. Specifically, mammal refers to a vertebrate animal that is human and non-human, which are members of the taxonomic class Mammalia. Non-exclusive examples of non-human mammals include companion animals and livestock. Non-exclusive examples of a companion animal include: dog, cat, and horse. Non-exclusive examples of livestock include: swine, goat, sheep, deer, and cattle, including bison. Preferred livestock is cattle. Specifically, fish refers to the taxonomic class Chondrichthyes (cartilaginous fishes, e.g., sharks and rays) and Osteichthyes (bony fishes) which live in water, have gills or mucus-covered skin for respiration, fins, and may have scales. Non-exclusive examples of fish include shark, salmon, trout, whitefish, catfish, tilapia, sea bass, tuna, halibut, turbot, flounder, sole, striped bass, eel, yellowtail, grouper, and the like.

“Infestation”, as used herein, unless otherwise indicated, refers to the state or condition of having parasites on the body. Furthermore, the infestation may lead to an infection on or in the animal, which may be microbial, viral, or fungal.

“Parasite(s)”, as used herein, unless otherwise indicated, refers to ectoparasites. Ectoparasites are organisms of the Arthropoda phylum (arachnids and insects) which feed through or upon the skin of its host. Preferred arachnids are of the order Acarina, e.g., ticks and mites. Preferred insects are of the Order Diptera which include midges, fleas, mosquitos, biting flies (e.g., stable fly, horn fly, blow fly (e.g., cochliomyia), horse fly, sand fly, and the like), and lice. Parasites also encompasses the different life stages of the ectoparasite, including eggs, pupae, and larvae which feed on or in the body.

“Therapeutically effective amount”, as used herein, unless otherwise indicated, refers to an amount of one of the fluorinated cyclopropyl phenylpyrazoles of the present invention that (i) treat or prevent the particular parasitic infestation, (ii) attenuates, ameliorates, or eliminates one or more symptoms of the particular parasitic infestation, or (iii) prevents or delays the onset of one or more symptoms of the particular parasitic infestation described herein. A dose range of about 1 to 5 mg/kg is contemplated to be a therapeutically effective dose. More preferred is a 2 mg/kg dose.

“Treatment”, “treating”, and the like, as used herein, unless otherwise indicated, refers to reversing, alleviating, or inhibiting the parasitic infestation. As used herein, these terms also encompass, depending on the condition of the animal preventing the onset of a disorder or condition, or of symptoms associated with a disorder or condition, including reducing the severity of a disorder or condition or symptoms associated therewith prior to affliction with said infection or infestation. Thus, treatment can refer to administration of the veterinary composition of the present invention to an animal that is not at the time of administration afflicted with the parasitic infestation, for example, as prophylactic treatment. Treating also encompasses preventing the recurrence of an infestation or of symptoms associated therewith as well as references to “control” (e.g., kill, repel, expel, incapacitate, deter, eliminate, alleviate, minimize, and eradicate).

“Veterinary acceptable” as used herein, unless otherwise indicated, indicates that the substance or composition must be compatible chemically and/or toxicologically with the other ingredients comprising the veterinary composition and/or the animal being treated therewith. Veterinary acceptable is equivalent to pharmaceutically acceptable.

### DETAILED DESCRIPTION

Figure Description:

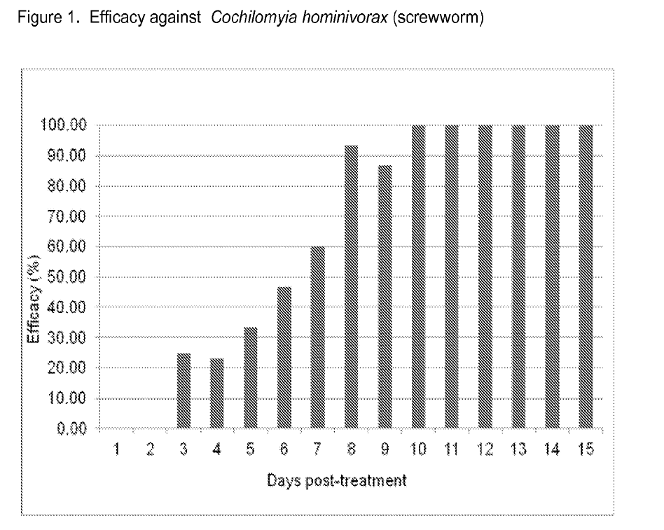

FIG. 1: Efficacy against Cochilomyia hominivorax (screwworm)

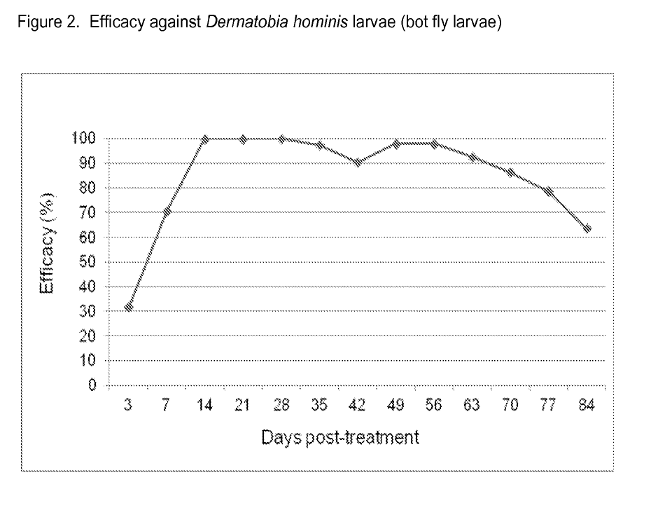

FIG. 2: Efficacy against Dermatobia hominis larvae (bot fly larvae)

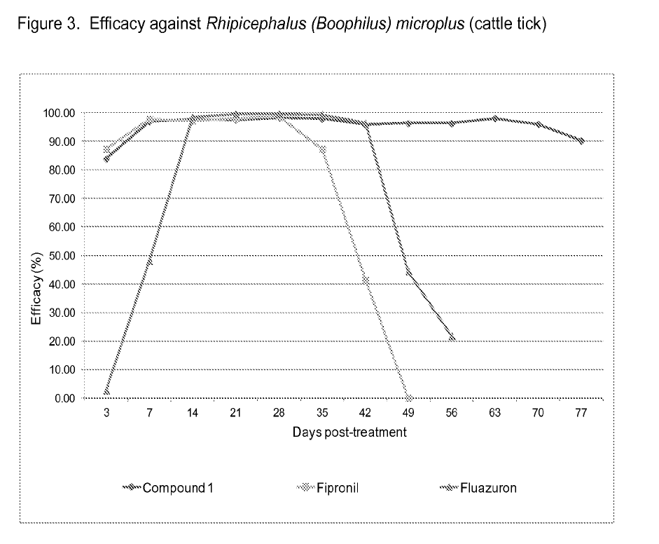

FIG. 3: Efficacy against Rhipicephalus (Boophilus) microplus (cattle tick)

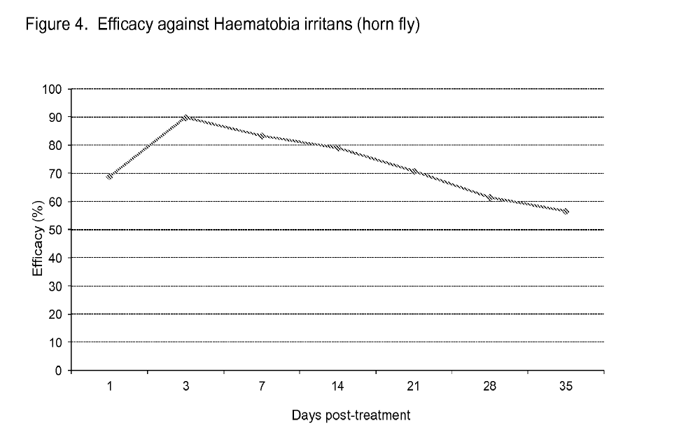

FIG. 4: Efficacy against Haematobia irritans (horn fly)

It is to be understood by one of ordinary skill in the art that the present discussion is a description of exemplary embodiments only and is not intended as limiting the broader aspects of the present invention, which broader aspects are embodied in the exemplary construction. In fact, it will be apparent to those skilled in the art that various modifications and variations can be made in the present invention without departing from the scope or spirit of the invention. For instance, features illustrated or described as part of one embodiment can be used in another embodiment to yield a still further embodiment. It is intended that the present invention cover such modifications and variations as come within the scope of the appended claims and their equivalents.

The present invention provides for a veterinary composition for the treatment of a parasitic infestation in an animal which comprises a veterinary effective amount of a fluorinated cyclopropylphenylpyrazole compound selected from 5-amino-1-[2,6-dichloro-4-(trifluoromethyl)phenyl]-4-[2,2-difluoro-1-(trifluoromethyl)cyclopropyl]-1H-pyrazole-3-carbonitrile; 5-amino-1-[2,6-dichloro-4-(trifluoromethyl)phenyl]-4-[1-(difluoromethyl)-2,2-difluorocyclopropyl]-1H-pyrazole-3-carbonitrile; 5-amino-4-{1-[chloro(fluoro)methyl]-2,2-difluorocyclopropyl}-1-[2,6-dichloro-4-(trifluoro-methyl)phenyl]-1H-pyrazole-3-carbonitrile; 5-amino-1-[2,6-dichloro-4-(trifluoro-methyl)phenyl]-4-[1-(difluoromethyl)-2,2,3,3-tetrafluorocyclopropyl]-1H-pyrazole-3-carbonitrile; and 5-amino-1-[2,6-dichloro-4-(trifluoromethyl)phenyl]-4-[2,2,3,3-tetrafluoro-1-(trifluoromethyl)cyclopropyl]-1H-pyrazole-3-carbonitrile; stereoisomer thereof, or a veterinary acceptable salt thereof. The veterinary acceptable composition further comprises at least one veterinary acceptable carrier, or a mixture thereof, and optionally, at least one antioxidant.

The fluorinated cyclopropylphenylpyrazole compounds can be synthesized according to procedures described in WO2005/060749.

It is to be understood that the fluorinated cyclopropylphenylpyrazole compounds may contain one or more asymmetric carbon (chiral) atoms, thus compounds of the invention can exist as two or more stereoisomers. Included within the scope of the present invention are all stereoisomers such as enantiomers (e.g. S and R enantiomers) and diasteromers, all geometric isomers and tautomeric forms of the fluorinated cyclopropylphenylpyrazole compounds. Compound 1 of the present invention is a racemate, and includes the (S) and (R) enantiomers. The (S) and (R) enantiomers can be separated, as described below, and can be administered to an animal as enantiomerically pure compounds in the veterinary composition of the present invention. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). Alternatively, the racemate (or a racemic precursor) may be reacted with a suitable optically active compound, for example, an alcohol. Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% isopropanol, typically from 2 to 20%, and from 0 to 5% of an alkylamine, typically 0.1% diethylamine. Concentration of the eluate affords the enriched mixture.

Veterinary compositions suitable for the delivery of compounds of the present invention and methods for their preparation will be readily apparent to those skilled in the art. Such compositions and methods for their preparation may be found, for example, in ‘Remington's Pharmaceutical Sciences’, 19th Edition (Mack Publishing Company, 1995).

In the present invention, the veterinary acceptable carrier comprises at least one triglyceride, at least one solvating agent, and mixtures thereof. The solvating agents include fatty acid esters or alcohols. The solvating agents are generally neutral and nonpolar thereby providing superior solvent characteristics. Non-exclusive examples of the solvating agents include: tricaprylin, ethyl oleate, caprylic/capric triglyceride, triacetin, caprylic/capric/linoleic triglyceride, caprylic/capric/succinic triglyceride, propylene glycol dicaprylate/dicaprate, butylene glycol dicaprylate/dicaprate, diacetylated monoglycerides, oleyl erucate, cetearyl ethylhexanoate, isopropylmyristate, decyloleate, glyceryl trihexanoate, triheptanoin, benzyl benzoate, caprylic alcohol, capric alcohol, lauryl alcohol, myristyl alcohol, cetyl alcohol, stearyl alcohol, cetearyl alcohol, and the like. The veterinary composition of the present invention further comprises a mixture of solvating agents.

The veterinary acceptable carrier further comprises a triglyceride. The term triglyceride also encompasses the mono- and di-glycerides. Further, triglyceride encompasses the naturally derived and semi-synthetic/synthetic oils. Non-exclusive examples of triglycerides include: castor oil, cottonseed oil, sesame oil, linseed oil, safflower oil, peanut oil, soybean oil, coconut oil, olive oil, corn oil, almond oil, vegetable oil, glyceryl stearates, glyceryl hexanoates, caprylic/capric glycerides, glyceryl cocoate, caprylic glycerides, glyceryl monooleate, glyceryl ricinoleate, capric glycerides, and the like. The veterinary composition of the present invention further comprises a mixture of triglycerides.

The veterinary acceptable carrier further comprises at least one solvating agent, or a mixture thereof, and at least one triglyceride, or a mixture thereof.

The veterinary acceptable carrier further comprises a co-solvent. Non-exclusive examples of co-solvents include: di-glycol monoalkyl ethers, for example, di(C₂₋₄ glycol)mono(C₁₋₄alkyl) ether (e.g., diethylene glycol monomethyl ether, dipropylene glycol monomethyl ether, diethylene glycol monoethyl ether, and the like), and alcohols (ethanol, benzyl alcohol, n-butanol, propanol, and the like) and esters thereof. The veterinary composition of the present invention further comprises a mixture of co-solvents.

The amounts of the various veterinary acceptable carriers can range from about 0-60% of a triglyceride and 0-100% of a solvating agent. The preferred range for the triglyceride is about 5-50%. More preferred, the triglyceride ranges from about 5-35%. Even more preferred, the triglyceride ranges from about 5-20%. Most preferred, the triglyceride is about 10%. The preferred range for the solvating agent is about 40-100%. More preferred, the range for the solvating agent is about 50-95%. Even more preferred, the range for the solvating agent is about 70-95%. Even more preferred, the range for the solvating agent is about 80-95%. Most preferred, the range for the solvating agent is about 85-90%. The preferred amounts for the co-solvents range from about 0-15%. Even more preferred, the cosolvent ranges from about 3-10%. The amounts of an antioxidant(s) included in the veterinary composition ranges from about 0 to 0.5%. More preferred, the antioxidant(s) range from about 0.01 to about 0.3%. Even more preferred, the antioxidant(s) range from about 0.01 to about 0.1%. Even more preferred, the antioxidant(s) range from about 0.01 to about 0.06%. Even more preferred, the antioxidant(s) range from about 0.01 to about 0.04%. Most preferred, the antioxidant(s) range from about 0.02 to about 0.03%. The percent ranges described herein for the the veterinary acceptable carriers and antioxidants refer to normalized weight percents.

Non-limiting examples of a veterinary acceptable composition comprising a fluorinated cyclopropylphenylpyrazole compound include the non-limiting examples of veterinary acceptable carriers: castor oil and propylene glycol dicaprylate/dicaprate; cottonseed oil and ethyl oleate and benzyl benzoate; cotton seed oil and propylene glycol dicaprylate/dicaprate; safflower oil or sesame oil or olive oil and ethyl oleate and benzyl benzoate; safflower oil or sesame oil or corn oil and propylene glycol dicaprylate/dicaprate; and the like.

In another aspect of the present invention, the composition further comprises an antioxidant. Non-exclusive antioxidants include vitamin C (ascorbic acid), vitamin E (tocopherol), vitamin E derivatives, butylated hydroxanisole (BHA), and butylated hydroxytoluene (BHT), citric acid, propyl gallate, and the like. Preferred antioxidants include BHA and BHT.

Such compositions are prepared in a conventional manner in accordance with standard medicinal or veterinary practice.

The amounts of these fluorinated cyclopropylphenylpyrazole compounds are easily determined by a skilled artisan and further depend on the dose amount and dose volume of the final composition. Representative amounts of a veterinary effective amount of a fluorinated cyclopropylphenylpyrazole ranges from about 0.5 mg/kg to about 5 mg/kg, with a preferred range of about 1 mg/kg to about 3 mg/kg. The more preferred dose of a fluorinated cyclopropylphenylpyrazole is about 2 mg/kg. The preferred amount of a fluorinated cyclopropylphenylpyrazole in the composition ranges from about 50 mg/mL to about 200 mg/mL. More preferred, the amount of the fluorinated cyclopropylphenylpyrazole is about 75 mg/mL to about 150 mg/mL. Even more preferred, the amount of the fluorinated cyclopropyl-phenylpyrazole is about 100 mg/mL. Dose volume for the final composition ranges from about 0.01 mL/kg to about 0.05 mL/kg of animal body weight. More preferred, dose volume ranges from about 0.01 mL/kg to about 0.04 mL/kg of animal body weight. Even more preferred, dose volume is about 0.01 mL/kg to about 0.03 mL/kg of animal body weight. Even more preferred, dose volume is about 0.01 mL/kg to about 0.02 mL/kg of animal body weight. Most preferred, dose volume is 0.02 mL/kg of animal body weight.

The fluorinated cyclopropylphenylpyrazole compositions of the present invention are useful as parasiticides for the control and treatment of parasitic infestations in an animal. The veterinary compositions of the present invention have utility as a parasiticide, in particular, as an acaricide and insecticide. They may, in particular, be used in the fields of veterinary medicine, livestock husbandry and the maintenance of public health: against acarids and insects which are parasitic upon animals, particularly domestic animals such as dogs, cats, cattle, sheep, goats, horses, llamas, bison, and swine, more particularly cattle. Some non-limiting examples of acaride and insect parasites include: ticks (e.g., Ixodes spp., Rhipicephalus spp., Boophilus spp., Amblyomma spp., Hyalomma spp., Haemaphysalis spp., Dermacentor spp., Ornithodorus spp., and the like); mites (e.g., Damalinia spp., Dermanyssus spp., Sarcoptes spp., Psoroptes spp., Eutrombicula spp., Chorioptes spp., Demodex spp., and the like); chewing and sucking lice (e.g., Damalinia spp., Linognathus spp., and the like); fleas (e.g., Siphonaptera spp., Ctenocephalides spp., and the like); and biting flies, mosquitos, and midges (e.g., Order Diptera; Aedes spp., Anopheles spp., Tabanidae spp., Haematobia spp., Stomoxys spp., Dermatobia spp., Simuliidae spp., Ceratopogonidae spp., Psychodidae spp., Cochliomyia spp., Muscidae spp., Hypoderma spp., Gastrophilus spp., Simulium spp., Hemiptera spp., Phthiraptera spp., Damalinia spp., Linognathus spp., Periplaneta spp., Blatella spp., Hymenoptera spp., and the like).

The veterinary compositions of the present invention are of particular value in the control of ectoparasites and insects which are injurious to, or spread or act as vectors of diseases in animals, for example those described herein, and more especially in the control of ticks, mites, lice, fleas, midges and biting, nuisance flies. They are particularly useful in controlling acarids and insects which feed on the skin or tissue or suck the blood of the animal, for which purpose they may be administered subcutaneously.

Systemic delivery of the fluorinated cyclopropylphenylpyrazole via subcutaneous injection ensures the entire treatment dose is delivered to the animal, with no loss due to environmental conditions (e.g., rain) that can adversely affect topical delivery. Upon injection, the fluorinated cyclopropylphenylpyrazole is stored in the body fat of the animal and is slowly released to the animal's circulatory system and skin, providing prolonged ectoparasitic control. The fluorinated cyclopropylphenylpyrazole compound binds tightly to ligand-gated chloride channels, in particular those gated by the neurotransmitter gamma-aminobutyric acid (GABA), thereby blocking pre- and post-synaptic transfer of chloride ions across cell membranes in insects and acarids when exposed by ingestion or contact. This mechanism of action results in lethal uncontrolled activity of the central nervous system of insects and acarids yielding highly efficacious control. The fluorinated cyclopropylphenylpyrazole injectable composition has excellent syringability, stability, and efficacy.

The veterinary compositions of the present invention also have value for the treatment and control of the various lifecycle stages of arachnids and insects, including egg, nymph, larvae, juvenile and adult stages.

The present invention also relates to a method of administering a veterinary composition of the present invention to an animal in good health comprising the application to said animal to reduce or eliminate the potential for human parasitic infection or infestation from parasites carried by the animal and to improve the environment in which the animals and humans inhabit.

### Composition Examples

In the following composition tables, FPP refers to fluorinated cyclopropylphenylpyrazole. With the examples provided, the cottonseed oil and/or castor oil can be replaced or mixed with any other triglyceride as described herein. Similarly, the solvating agent can be replaced or mixed with any other solvating agent described herein. Similarly, the antioxidant can be replaced or mixed with any other antioxidant described herein. Further, at least one co-solvent can be added to the composition. The solids are exemplified as mg/mL and normalized weight %, and liquids are exemplified as mL/mL and normalized weight %. The following non-limiting veterinary composition examples include:

### Formula 1

| | | | | Normalized |
|---|---|---|---|---|
| Component | Function | Component Amount | | Weight % |
| FPP | active | 100 | mg/mL | 10 |
| BHA | antioxidant | 0.3 | mg/mL | 0.03 |
| Cottonseed oil | carrier | 0.1 | mL/mL | 9 |
| Propylene glycol | carrier | qs 1 | mL | 81 |
| dicaprylate/dicaprate | | | | |

### Formula 2

| | | | | Normalized |
|---|---|---|---|---|
| Component | Function | Component Amount | | Weight % |
| FPP | active | 100 | mg/mL | 10 |
| BHT | antioxidant | 0.3 | mg/mL | 0.03 |
| Cottonseed oil | carrier | 0.1 | mL/mL | 9 |
| Propylene glycol | carrier | qs 1 | mL | 81 |
| dicaprylate/dicaprate | | | | |

### Formula 3

| | | | | Normalized |
|---|---|---|---|---|
| Component | Function | Component Amount | | Weight % |
| FPP | active | 100 | mg/mL | 10 |
| BHA | antioxidant | 0.3 | mg/mL | 0.03 |
| Cottonseed oil | carrier | 0.4 | mL/mL | 36 |
| Ethyl oleate/benzyl | carrier | qs 1 | mL | 54 |
| benzoate (40/20) | | | | |

### Formula 4

| | | | | Normalized |
|---|---|---|---|---|
| Component | Function | Component Amount | | Weight % |
| FPP | active | 100 | mg/mL | 10 |
| BHT | antioxidant | 0.3 | mg/mL | 0.03 |
| Cottonseed oil | carrier | 0.1 | mL/mL | 9 |
| Ethyl oleate/benzyl | carrier | qs 1 | mL | 81 |
| benzoate (40/20) | | | | |

### Formula 5

| | | | | Normalized |
|---|---|---|---|---|
| Component | Function | Component Amount | | Weight % |
| FPP | active | 100 | mg/mL | 10 |
| BHA | antioxidant | 0.3 | mg/mL | 0.03 |
| Castor oil | carrier | 0.1 | mL/mL | 10 |
| Propylene glycol | carrier | qs 1 | mL | 80 |
| dicaprylate/dicaprate | | | | |

### Formula 6

| | | | | Normalized |
|---|---|---|---|---|
| Component | Function | Component Amount | | Weight % |
| FPP | active | 100 | mg/mL | 10 |
| BHT | antioxidant | 0.3 | mg/mL | 0.03 |
| Castor oil | carrier | 0.1 | mL/mL | 10 |
| Propylene glycol | carrier | qs 1 | mL | 80 |
| dicaprylate/dicaprate | | | | |

### Formula 7

| | | | | Normalized |
|---|---|---|---|---|
| Component | Function | Component Amount | | Weight % |
| FPP | active | 50 | mg/mL | 5 |
| Castor oil | carrier | 0.6 | mL/mL | 60 |
| Ethyl oleate | carrier | qs 1 | mL | 35 |

### Formula 8

| | | | | Normalized |
|---|---|---|---|---|
| Component | Function | Component Amount | | Weight % |
| FPP | active | 100 | mg/mL | 10 |
| Castor oil | carrier | 0.4 | mL/mL | 40 |
| Propylene glycol | carrier | qs 1 | mL | 50 |
| dicaprylate/dicaprate | | | | |

### Formula 9

| | | | | Normalized |
|---|---|---|---|---|
| Component | Function | Component Amount | | Weight % |
| FPP | active | 100 | mg/mL | 10 |
| Caprylic/capric | carrier | qs 1 | mL | 90 |
| triglyceride | | | | |

### Formula 10

| | | | | Normalized |
|---|---|---|---|---|
| Component | Function | Component Amount | | Weight % |
| FPP | active | 200 | mg/mL | 20 |
| Triacetin | carrier | 0.6 | mL/mL | 60 |
| Propylene glycol | carrier | qs 1 | mL | 20 |
| dicaprylate/dicaprate | | | | |

### Formula 11

| | | | | Normalized |
|---|---|---|---|---|
| Component | Function | Component Amount | | Weight % |
| FPP | active | 200 | mg/mL | 20 |
| Triacetin | carrier | 0.55 | mL/mL | 55 |
| Ethanol | co-solvent | 0.05 | mL/mL | 5 |
| Propylene glycol | carrier | qs 1 | mL | 20 |
| dicaprylate/dicaprate | | | | |

### Biological

The fluorinated cyclopropylphenylpyrazole, 5-amino-1-[2,6-dichloro-4-(trifluoromethyl)phenyl]-4-[2,2-difluoro-1-(trifluoromethyl)cyclopropyl]-1H-pyrazole-3-carbonitrile (Compound 1) was used to conduct numerous in-vivo studies. These studies assessed the pharmacokinetics of different veterinary compositions and doses of the compound in cattle as well as the efficacy of the compound in a veterinary composition (Formula 1) against ticks, bot fly, horn fly, and screwworm.

The pharmacokinetic profiles of Compound 1 prepared in accordance with Formula 1, Formula 7, Formula 8, Formula 9, and Formula 10, was assessed in mixed-breed cattle. Cattle received a single 2 mg/kg dose of Compound 1 via subcutaneous injection. A validated HPLC-MS/MS test method was used for plasma sample analysis. Pharmacokinetic parameters were calculated using non-compartmental techniques, nominal sample times, nominal doses and the average value of duplicate sample analysis. The mean pharmacokinetic results are presented in Table 1. The observed pharmacokinetic profiles demonstrate prolonged absorption and elimination of Compound 1, with good exposure levels.

TABLE 1
Compound 1 Plasma Pharmacokinetics
PK parameters

| | Formula | Formula | Formula | Formula | Formula |
|---|---|---|---|---|---|
| PK parameter | 1 | 7 | 8 | 9 | 10 |
| Cmax (ng/mL) | 75 | 78 | 105 | 61 | 76 |
| Tmax (days) | 16 | 15 | 13 | 18 | 13 |
| AUC | 3950 | 1700 | 2180 | 1380 | 1710 |
| (day · ng/mL) | (AUC | (AUC | (AUC | (AUC | (AUC |
| | 0-∞) | 0-28 d) | 0-28 d) | 0-28 d) | 0-28 d) |
| T½ (days) | 23 | — | — | — | — |
| MRT (days) | 48 | — | — | — | — |

### Screwworm

The efficacy of Compound 1 in Formula 1 against infestation of Cochliomyia hominivorax (screwworm) in post-castration scrotal wounds of cattle was investigated in Brazil. The cattle were mixed-breed and 15 animals were included in the test group. Animals received a single 2.0 mg/kg subcutaneous injection on the day of castration. Untreated animals (15) were used as controls. Mean efficacy results are shown in FIG. 1. Results showed efficacy reaching 100% by day 10 post-treatment, and holding at 100% through day 15, indicating the veterinary composition can be an aid in the control of scrotal myiasis (C. hominivorax larvae) in recently castrated cattle.

### Bot Fly Larvae

The efficacy of Compound 1 in Formula 1 was investigated against Dermatobia hominis larvae (bot fly larvae) in naturally infested pasture cattle in Brazil. The cattle were mixed-breed and 10 animals were included in the test group. Each test animal received a single 2.0 mg/kg subcutaneous injection. Untreated animals (10) were used as controls. Mean efficacy results are shown in FIG. 2. Results showed efficacy >90% over days 14-60, indicating the veterinary composition can be recommended for the treatment and control of D. hominis larvae in cattle.

### Tick Study

The efficacy of Compound 1 in Formula 1 was investigated on pastured cattle naturally infested with Rhipicephalus (Boophilus) microplus (southern cattle ticks), in the southeast region of Brazil. The cattle were mixed-breed. The study used eight animals per test group. Animals received a single 2.0 mg/kg subcutaneous injection. For comparison, commercially available tick control products were included in the study. The 1% fipronil topical product, Topline®, and the 2.5% fluazuron product, Acatak®, were dosed according to manufacturer's instructions. Untreated animals were used as controls. Mean efficacy results are shown in FIG. 3. The tick results showed efficacy 95% over days 7-60 post-treatment, indicating the veterinary composition can be recommended for treatment and control of Rhipicephalus (Boophilus) microplus cattle ticks, and has a duration of efficacy greater than that of current fipronil and fluazuron commercial products.

### Horn Fly

The efficacy of Compound 1 in Formula 1 was investigated for efficacy against Haematobia irritans (horn fly) in naturally infested pasture cattle in Brazil. The cattle were mixed-breed, 15 animals were included in the test group. Each test animal received a single 2.0 mg/kg subcutaneous injection. Untreated animals (15) were used as controls. Mean efficacy results are shown in FIG. 4. Results showed efficacy >80% over days 3-14, indicating the veterinary composition can be recommended for the treatment and control of H. irritans (horn fly) infestation.

Overall, clinical efficacy of Compound 1 in Formula 1 in cattle given a single 2 mg/kg subcutaneous injection against tick, bot fly, horn fly, and screwworm is presented in Table 2.

TABLE 2
Summary of Efficacy Results of Compound 1 (Formula 1)
Compound 1 Efficacy Data Summary

| | % efficacy | | | |
|---|---|---|---|---|
| | Rhipicephalus | | | |
| | (Boophilus) | Cochiliomyia | Dermatobia | Haematobia |
| Days post | microplus | hominivorax | hominis larvae | irritans |
| treatment | (cattle tick) | (screwworm) | (bot fly larvae) | (horn fly) |
| 3 | 79 | 25 | 32 | 90 |
| 7 | 97 | 60 | 71 | 83 |
| 10 | — | 100 | — | — |
| 14 | 97 | 100 | 100 | 79 |
| 21 | 98 | — | 100 | 70 |
| 28 | 98 | — | 100 | 62 |
| 35 | 95 | — | 98 | 57 |
| 42 | 95 | — | 90 | — |
| 49 | 95 | — | 98 | — |
| 56 | 95 | — | 98 | — |
| 63 | 95 | — | 93 | — |
| 70 | 89 | — | 86 | — |
| 77 | 77 | — | 79 | — |
| 84 | 69 | — | 63 | — |

### Veterinary Composition Stability

Stability of Compound 1 in Formula 1 was assessed. The samples were placed on accelerated (40° C./75% relative humidity) stability for up to 6-months. As shown in Table 3, Compound 1 is stable for at least 6-months in Formula 1 under accelerated conditions. Therefore, it is contemplated that the Formula 1 composition will have an extended shelf-life.

TABLE 3
Compound 1 Stability in Formula 1

| | Compound 1 | Density |
|---|---|---|
| Months | (mg/mL) | (g/mL) |
| 0 | 99 | 0.963 |
| 1 | 99 | 0.962 |
| 2 | 101 | 0.962 |
| 3 | 99 | 0.962 |
| 6 | 98 | 0.963 |

We claim:

1. A veterinary composition comprising:
a) a fluorinated cyclopropylphenylpyrazole compound selected from
5-amino-1-[2,6-dichloro-4-(trifluoromethyl)phenyl]-4-[2,2-difluoro-1-(trifluoromethyl)cyclopropyl]-1H-pyrazole-3-carbonitrile;
5-amino-1-[2,6-dichloro-4-(trifluoromethyl)phenyl]-4-[1-(difluoromethyl)-2,2-difluorocyclopropyl]-1H-pyrazole-3-carbonitrile;
5-amino-4-{1-[chloro(fluoro)methyl]-2,2-difluorocyclopropyl}-1-[2,6-dichloro-4-(trifluoro-methyl)phenyl]-1H-pyrazole-3-carbonitrile;
5-amino-1-[2,6-dichloro-4-(trifluoro-methyl)phenyl]-4-[1-(difluoromethyl)-2,2,3,3-tetrafluorocyclopropyl]-1H-pyrazole-3-carbonitrile; and
5-amino-1-[2,6-dichloro-4-(trifluoromethyl)phenyl]-4-[2,2,3,3-tetrafluoro-1-(trifluoromethyl)cyclopropyl]-1H-pyrazole-3-carbonitrile; stereoisomer thereof, or a veterinary acceptable salt thereof;
b) a triglyceride;
c) a solvating agent; and optionally,
d) an antioxidant.

2. The veterinary composition of claim 1 wherein the triglyceride is selected from the group consisting of cottonseed oil, castor oil, sesame oil, linseed oil, safflower oil, peanut oil, and soybean oil, or a mixture thereof.

3. The veterinary composition of claim 2 wherein the solvating agent is selected from the group selected from ethyl oleate, caprylic/capric triglyceride, caprylic/capric/linoleic triglyceride, caprylic/capric/succinic triglyceride, propylene glycol dicaprylate/dicaprate, triacetin, benzyl benzoate, or a mixture thereof.

4. The veterinary composition of claim 3 wherein the solvating agent is selected from the group consisting of ethyl oleate, triacetin, and propylene glycol dicaprylate/dicaprate, or a mixture thereof, and the triglyceride is cotton seed oil.

5. The veterinary composition of claim 4 further comprising a co-solvent.

6. The veterinary composition of claim 5 wherein said co-solvent is selected from ethanol, a di-glycol monoalkyl ether, and benzyl alcohol.

7. The veterinary composition of claim 4 wherein the solvating agent is propylene glycol dicaprylate/dicaprate and further comprising an antioxidant.

8. The veterinary composition of claim 7 wherein said antioxidant is butylated hydroxyanisole, butylated hydroxytoluene, or a mixture thereof.

9. The veterinary composition of claim 8 wherein said fluorinated cyclopropylphenylpyrazole compound is 5-amino-1-[2,6-dichloro-4-(trifluoromethyl)phenyl]-4-[2,2-difluoro-1-(trifluoromethyl)cyclopropyl]-1H-pyrazole-3-carbonitrile, or stereoisomer thereof, or veterinary acceptable salt thereof.

10. A veterinary composition comprising a) a fluorinated cyclopropylphenylpyrazole compound that is 5-amino-1-[2,6-dichloro-4-(trifluoromethyl)phenyl]-4-[2,2-difluoro-1-(trifluoromethyl)cyclopropyl]-1H-pyrazole-3-carbonitrile, or stereoisomer thereof, or veterinary acceptable salt thereof; b) a triglyceride selected from cottonseed oil, castor oil, sesame oil, linseed oil, safflower oil, peanut oil, and soybean oil, or a mixture thereof; c) a solvating agent selected from the group consisting of ethyl oleate, triacetin, and propylene glycol dicaprylate/dicaprate, or a mixture thereof; and d) an antioxidant.

11. A method of treating an animal with a parasitic infestation comprising administering to said animal in need thereof a veterinary composition comprising:
a) a fluorinated cyclopropylphenylpyrazole compound selected from
5-amino-1-[2,6-dichloro-4-(trifluoromethyl)phenyl]-4-[2,2-difluoro-1-(trifluoromethyl)cyclopropyl]-1H-pyrazole-3-carbonitrile;
5-amino-1-[2,6-dichloro-4-(trifluoromethyl)phenyl]-4-[1-(difluoromethyl)-2,2-difluorocyclopropyl]-1H-pyrazole-3-carbonitrile;
5-amino-4-{1-[chloro(fluoro)methyl]-2,2-difluorocyclopropyl}-1-[2,6-dichloro-4-(trifluoro-methyl)phenyl]-1H-pyrazole-3-carbonitrile;
5-amino-1-[2,6-dichloro-4-(trifluoro-methyl)phenyl]-4-[1-(difluoromethyl)-2,2,3,3-tetrafluorocyclopropyl]-1H-pyrazole-3-carbonitrile; and
5-amino-1-[2,6-dichloro-4-(trifluoromethyl)phenyl]-4-[2,2,3,3-tetrafluoro-1-(trifluoromethyl)cyclopropyl]-1H-pyrazole-3-carbonitrile, stereoisomer thereof, or a veterinary acceptable salt thereof;
b) a triglyceride;
c) a solvating agent; and optionally,
d) an antioxidant.

12. The method of claim 11, wherein said fluorinated cyclopropylphenylpyrazole compound is 5-amino-1-[2,6-dichloro-4-(trifluoromethyl)phenyl]-4-[2,2-difluoro-1-(trifluoromethyl)cyclopropyl]-1H-pyrazole-3-carbonitrile, stereoisomer thereof, or veterinary acceptable salt thereof, and further comprising an antioxidant.

13. The method of claim 12, wherein the triglyceride is cotton seed oil, the solvating agent is propylene glycol dicaprylate/dicaprate, and the antioxidant is butylated hydroxyanisole, butylated hydroxytoluene, or a mixture thereof.

14. The method of claim 13 further comprising a co-solvent selected from ethanol, a di-glycol monoalkyl ether, and benzyl alcohol.

15. The method of claim 11 wherein the veterinary composition is administered by injection and the animal is cattle, sheep, swine, goat, or horse.

16. The method of claim 15 wherein the animal is cattle.
